(19) United States
(12) Patent Application Publication
(10) Pub. No.: US 2024/0170120 A1
(43) Pub. Date: May 23, 2024

(54) HEALTH INFORMATION GENERATING APPARATUS, HEALTH INFORMATION GENERATING METHOD, AND NON-TRANSITORY COMPUTER-READABLE MEDIUM

(71) Applicants: NEC Corporation, Minato-ku, Tokyo (JP); NEC PLATFORMS, LTD., Kanagawa (JP)

(72) Inventors: Hiroaki YAMAMOTO, Tokyo (JP); Kiichi Kanoo, Kanagawa (JP); Yang Yang, Tokyo (JP); Jingjing Ye, Tokyo (JP); Seisho Kuba, Tokyo (JP)

(73) Assignees: NEC Corporation, Minato-ku , Tokyo (JP); NEC Platforms, Ltd., Kawasaki-shi, Kanagawa (JP)

(21) Appl. No.: 18/282,858

(22) PCT Filed: Mar. 22, 2021
(86) PCT No.: PCT/JP2021/011655

§ 371 Date: Sep. 19, 2023

(51) Int. Cl. G16H 20/10

(52) U.S. Cl. CPC G16H 20/10

(57) ABSTRACT

A health information generating apparatus includes an acquiring unit, an attribute information generating unit, and a health information generating unit. The acquiring unit acquires customer identification information of a customer whose health information is to be generated, and acquires prescription information associated with the customer identification information. The attribute information generating unit generates attribute information of the customer by using the prescription information acquired by the acquiring unit. The attribute information includes information indicating a health state of the customer. The health information generating unit generates the above-described health information by using the attribute information generated by the attribute information generating unit. The health information relates to at least one of maintenance, improvement, and future change in health of the customer.

### TECHNICAL FIELD

The present invention relates to a health information generating apparatus, a health information generating method, and a program.

### BACKGROUND ART

A prescription describes information on a medication that a person should take. Then, Patent Document 1 describes deciding a type of advertisement to be printed on a receipt by using medication type information that determines a type of prescribed medication, and outputting a receipt on which an advertisement corresponding to the type of advertisement is printed.

### RELATED DOCUMENT

### Patent Document

Patent Document 1: Japanese Patent Application Publication No. 2006-154889

### SUMMARY OF INVENTION

### Technical Problem

As described above, information on a medication that a person should take is described in a prescription. Therefore, an inventor of the present invention considered that, by processing the information described in the prescription, information beneficial to a person for which the prescription is written can be provided to the person.

An object of the present invention is to generate, based on information described in a prescription, information being beneficial to a person for which the prescription is written.

### Solution to Problem

According to the present invention, a health information generating apparatus is provided, the apparatus including:
an acquiring unit that acquires prescription information being information described in a prescription for a customer;
an attribute information generating unit that generates, by using the prescription information, attribute information indicating an attribute of the customer; and
a health information generating unit that generates, by using the attribute information, health information being information on health of the customer, and outputs the health information.

According to the present invention, a health information generating method is provided, the method including,
performing by a computer:
acquiring processing of acquiring prescription information being information described in a prescription for a customer;
attribute information generating processing of generating, by using the prescription information, attribute information indicating an attribute of the customer; and
health information generating processing of generating, by using the attribute information, health information being information on health of the customer, and outputting the health information.

According to the present invention, a program is provided, the program causing a computer to include:
an acquiring function of acquiring prescription information being information described in a prescription for a customer;
an attribute information generating function of generating, by using the prescription information, attribute information indicating an attribute of the customer; and
a health information generating function of generating, by using the attribute information, health information being information on health of the customer, and outputting the health information.

### Advantageous Effects of Invention

According to the present invention, information being beneficial to a person for which the prescription is written can be generated, based on information described in a prescription.

### BRIEF DESCRIPTION OF THE DRAWINGS

The above-described object, another object, a feature, and an advantage are further clarified with a preferable example embodiment described in the following and the following drawings accompanying thereto.

FIG. 1 It is a diagram for describing a usage environment of a health information generating apparatus according to an example embodiment.

FIG. 2 It is a diagram illustrating one example of a functional configuration of the health information generating apparatus.

FIG. 3 It is a diagram illustrating one example of information stored in a personal information storage unit.

FIG. 4 It is a diagram illustrating a hardware configuration example of the health information generating apparatus.

FIG. 5 It is a flowchart illustrating a first example of processing performed by the health information generating apparatus.

FIG. 6 It is a diagram illustrating a modification example of FIG. 5.

### DESCRIPTION OF EMBODIMENTS

In the following description, an example embodiment of the present invention is described with reference to the drawings. Note that, in all of the drawings, a similar component is denoted with a similar reference sign, and description thereof will not be repeated as appropriate.

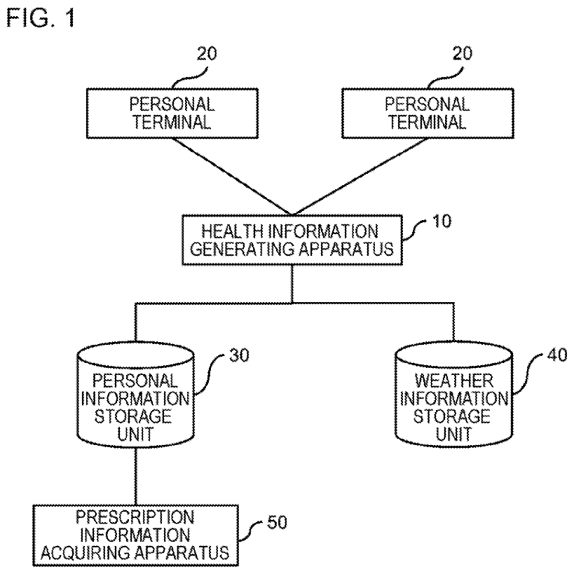

FIG. 1 is a diagram for describing a usage environment of a health information generating apparatus 10 according to an example embodiment. The health information generating apparatus 10 generates, by using information described in a prescription (hereinafter, described as prescription information), attribute information of a person for which the prescription is written (herein after, described as a customer), and generates, by using the attribute information, information on health of the customer (hereinafter, described as health information). The health information generating apparatus 10 transmits the generated health information to a personal terminal 20 possessed by the customer. A specific example of the health information is described later with reference to another drawing.

In the example illustrated in the present drawing, the prescription information is stored in a personal information storage unit 30. The personal information storage unit 30 stores the prescription information in association with information that distinguish a customer from one another (hereinafter, described as customer identification information). The customer identification information may be information including a name, or may be an ID assigned to a customer.

The prescription information is acquired by a prescription information acquiring apparatus 50. The prescription information acquiring apparatus 50 may acquire, via a storage medium or by communication, the prescription information and the customer identification information as electronic data. Alternatively, the prescription information acquiring apparatus 50 may generate the prescription information by reading a prescription printed on paper by using an optical character recognition (OCR) function. At this occasion, the prescription information acquiring apparatus 50 also reads the customer identification information described in the prescription. Then, the prescription information acquiring apparatus 50 stores the acquired prescription information in the personal information storage unit 30, in association with the acquired customer identification information. Herein, the personal information storage unit 30 stores not only most recent prescription information, but also previous prescription information. Therefore, personal information storage unit 30 stores a medication history of the customer.

The personal information storage unit 30 further stores a medical history and a product purchase history of a customer, in association with the customer identification information. When generating the health information, the information generating apparatus 10 uses the medical history and the product purchase history of a customer as necessary.

Further, when generating the health information, the health information generating apparatus 10 uses weather information as necessary. The weather information indicates, for example, a weather forecast for each area, and is stored in a weather information storage unit 40. The health information generating apparatus 10 reads a weather forecast for a location of the personal terminal 20 or a location of the prescription information acquiring apparatus 50 from the weather information storage unit 40, and uses the read weather forecast.

Note that, the personal information storage unit 30 and the weather information storage unit 40 may be a part of the health information generating apparatus 10.

Further, a transmission destination of the health information is not limited to the personal terminal 20. For example, when the prescription information acquiring apparatus 50 is located in a pharmacy, the health information may be a terminal installed in the pharmacy. In this case, the terminal may print the health information, display the health information on a display, or transmit the health information to the personal terminal 20 via short-range wireless communication.

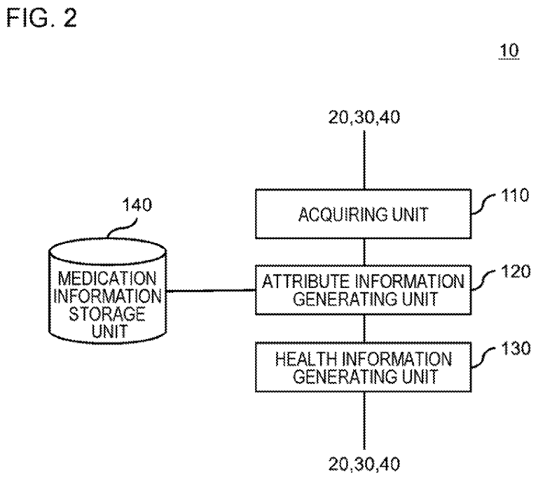

FIG. 2 is a diagram illustrating one example of a functional configuration of the health information generating apparatus 10. The health information generating apparatus 10 includes an acquiring unit 110, an attribute information generating unit 120, and a health information generating unit 130.

The acquiring unit 110 acquires prescription information from the personal information storage unit 30. For example, the acquiring unit 110 acquires customer identification information of a customer whose health information is to be generated, and acquires prescription information associated with the customer identification information. Further, the acquiring unit 110 acquires, as necessary, at least one of a medical history and purchase information associated with the acquired customer identification information, from the personal information storage unit 30. Further, the acquiring unit 110 acquires, as necessary, information indicating a current location (hereinafter, described as location information) of the personal terminal 20 from the personal terminal 20, and acquires weather information associated with the location information from the weather information storage unit 40. Note that, the location information may indicate a location of the prescription information acquiring apparatus 50. In this case, the personal information storage unit 30 stores the location information along with the prescription information.

The attribute information generating unit 120 generates attribute information of a customer by using the prescription information acquired by the acquiring unit 110. The attribute information includes information indicating a health state of a customer. For example, the attribute information generating unit 120 estimates a disease that a customer currently suffers from, based on a type of medication included in the prescription information, and adds a name of the disease (specifically, a disease name) to the attribute information. Note that, the attribute information generating unit 120 may estimate a symptom that a customer currently has and a physical constitution of the customer, based on the above-described type of medication, and add a result of the estimation to the attribute information. Herein, examples of the symptom include fever, a headache, nausea, indigestion, obesity, high blood pressure, low blood pressure, anemia, and the like. Further, examples of the physical constitution include a tendency to have a gastrointestinal problem, a tendency to have a headache, and the like. Further, when there is no specific problem, the attribute information is to be “no problem”.

Note that, the attribute information generating unit 120 may store the estimated disease in the personal information storage unit 30. The information is stored in the personal information storage unit 30 as a medical history of the customer.

In the example illustrated in the present drawing, the attribute information generating unit 120 generates attribute information by using information stored in a medication information storage unit 140. The medication information storage unit 140 stores, for each type of medication, at least one of a disease name, a symptom, and a physical constitution covered by the medication. Then, the attribute information generating unit 120 reads a disease name, a symptom, and a physical constitution associated with a medication indicated by the prescription information, from the medication information storage unit 140, and generates attribute information by using the disease name, the symptom, and the physical constitution. Note that, the medication information storage unit 140 may be a part of the health information generating apparatus 10, or may be provided external to the health information generating apparatus 10.

The health information generating unit 130 generates the above-described health information by using the attribute information generated by the attribute information generating unit 120, and transmits the generated health information to the personal terminal 20. When generating the health information, the attribute information generating unit 120 may use at least one of a purchase history, a medical history, a prescription history (specifically, a medication history), and weather information that are acquired by the acquiring unit 110.

The health information relates to at least one of maintenance, improvement, and future change in health of a customer. As one example, the health information may include, for example, information indicating a food and/or an ingredient (for example, a nutrient) that a customer should consume, or may include information indicating a food (which may be a specific product) and/or an ingredient (for example, salt or sugar) that the customer should not consume. Note that, a further example of the health information will be described later with reference to another drawing.

Note that, the health information generating unit 130 may generate, as part of the health information or in addition to the health information, recommendation information indicating at least one of an exercise that a customer should take or a diet that the customer should consume, and may transmit the recommendation information to the personal terminal 20. For example, the health information generating unit 130 estimates a disease that may occur to a customer in the future, by using at least one of a type of medication included in the prescription information and attribute information. Then, the health information generating unit 130 adds at least one of an exercise and a diet necessary to prevent the disease to the recommendation information.

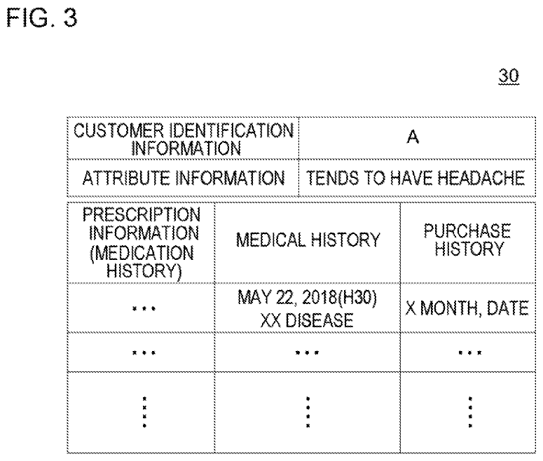

FIG. 3 is a diagram illustrating one example of information stored in the personal information storage unit 30. In the example illustrated in the present drawing, the personal information storage unit 30 stores, for each customer, customer identification information, prescription information, a medical history, and an item purchase history in association with one another.

Herein, as described above, the personal information storage unit 30 stores previous prescription information in addition to most recent prescription information. Further, a medical history to be stored in the personal information storage unit 30 is generated, for example, by the attribute information generating unit 120, but is not limited thereto.

Further, a purchase history to be stored in the personal information storage unit 30 is generated, for example, by a product registration apparatus or a payment apparatus installed in a pharmacy. At this occasion, the product registration apparatus or the payment apparatus also acquires customer identification information from, for example, a membership card, and stores the purchase history in the personal information storage unit 30, in association with the customer identification information. Note that, this processing may be performed by a product registration apparatus or a payment apparatus installed in another store (for example, a convenience store or a supermarket). The product registration apparatus or the payment apparatus is an apparatus, such as a point of sale (POS) terminal, for example, used for payment in a store.

Further, in the example illustrated in the present drawing, the personal information storage unit 30 also stores attribute information generated by the attribute information generating unit 120. The health information generating unit 130 of the health information generating apparatus 10 can also generate health information by using the attribute information.

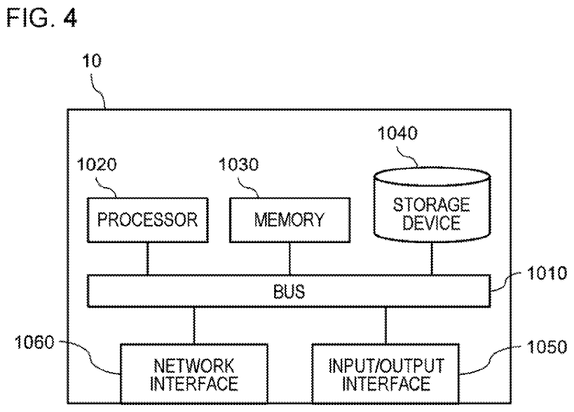

FIG. 4 is a diagram illustrating a hardware configuration example of the health information generating apparatus 10. The health information generating apparatus 10 includes a bus 1010, a processor 1020, a memory 1030, a storage device 1040, an input/output interface 1050, and a network interface 1060.

The bus 1010 is a data transmission path for the processor 1020, the memory 1030, the storage device 1040, the input/output interface 1050, and network interface 1060 to transmit and receive data to and from one another. However, a method for connecting the processor 1020 and the like to one another is not limited to bus connection.

The processor 1020 is a processor achieved with a central processing unit (CPU), a graphics processing unit (GPU), and the like.

The memory 1030 is a main storage apparatus achieved with a random access memory (RAM), and the like.

The storage device 1040 is an auxiliary storage device achieved with a hard disk drive (HDD), a solid state drive (SSD), a memory card, a read only memory (ROM), or the like. The storage device 1040 stores a program module that achieves each function (for example, the acquiring unit 110, the attribute information generating unit 120, and the health information generating unit 130) of the health information generating apparatus 10. The processor 1020 loads each of the program modules onto the memory 1030 and executes each of the program modules, and thereby each function related to the program module is achieved. Further, the storage device 1040 also functions as the medication information storage unit 140.

The input/output interface 1050 is an interface for connecting the health information generating apparatus 10 to various input/output devices.

The network interface 1060 is an interface for connecting the health information generating apparatus 10 to a network. The network is, for example, a local area network (LAN) or a wide area network (WAN). A method in which the network interface 1060 connects to the network may be wireless connection, or may be wired connection. The health information generating apparatus 10 may communicate with the personal terminal 20, the personal information storage unit 30, and the weather information storage unit 40, via the network interface 1060.

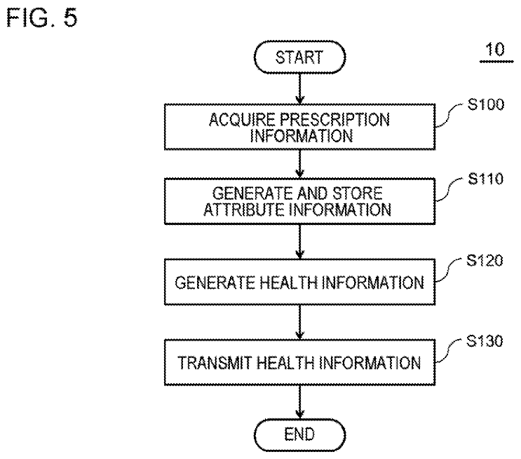

FIG. 5 is a flowchart illustrating a first example of processing performed by the health information generating apparatus 10. The health information generating apparatus 10 performs the processing illustrated in the present drawing, for example, when new prescription information is stored in the personal information storage unit 30.

The personal information storage unit 30 transmits newly stored prescription information to the acquiring unit 110 together with customer identification information associated with the prescription information (step S100). Then, the acquiring unit 110 acquires, as necessary, other information (for example, at least one of previous prescription information, a medical history, a purchase history, and location information of the prescription information acquiring apparatus 50) associated with the acquired customer identification information. Further, the acquiring unit 110 acquires, as necessary, weather information associated with the location information from the weather information storage unit 40.

Next, the attribute information generating unit 120 generates attribute information by using the prescription information acquired by the acquiring unit 110. One example of a generation method of the attribute information and one example of the attribute information are as described with reference to FIG. 2 (step S110). Herein, the attribute information generating unit 120 stores, as necessary, the generated attribute information in the personal information storage unit 30. Note that, this generation processing of attribute information may be performed by using the previous prescription information.

Next, the health information generating unit 130 generates health information by using the attribute information generated by the attribute information generating unit 120 (step S120). Note that, this generation processing of health information may be performed by using the medical history acquired by the acquiring unit 110. Further, the generation processing of health information may be performed by using the previous prescription information acquired by the acquiring unit 110.

For example, the health information generating unit 130 may determine, by using at least one of a disease name, a symptom, and a physical constitution included in the attribute information, information indicating a food material and/or a product for improving the at least one of the disease, the symptom, and the physical constitution, and add the information to the health information. As one example, when the attribute information indicates anemia, the health information includes information indicating a food material and/or a product containing iron. Note that, this processing may be performed by using the medical history stored in the personal information storage unit 30 instead of using attribute information.

Further, the health information generating unit 130 may determine, by using at least one of the disease name, the symptom, and the physical constitution included in the attribute information, an ingredient that may aggravate the at least one of the disease, the symptom, and the physical constitution, and add at least one of information indicating the ingredient, information indicating a food material containing the ingredient, and information indicating a product containing the ingredient, to the health information. Herein, determination of a food material and food is performed by using a database indicating ingredients contained in each food material and each product.

Further, the health information generating unit 130 may estimate a disease that may occur to a customer in the future, by using the prescription information, the attribute information, or the medical history. Specifically, the attribute information or the medical history indicates a disease that a customer currently suffers from or suffered in the past. Then, the current or past disease may be closely related to a disease that may occur to the customer in the future. By using this relationship, the health information generating unit 130 estimates a disease that may occur to a customer in the future. This estimation processing may be performed by using a model generated by machine learning, or may be performed by using a program prepared in accordance with a rule base. Further, the health information generating unit 130 may add a result of the estimation to the health information. Furthermore, the health information generating unit 130 may add information (for example, information indicating a desirable dietary and an exercise habit) beneficial for preventing a disease indicated by the estimation result to the health information.

Further, the health information generating unit 130 may generate the health information by further using the purchase history. As one example, the health information generating unit 130 generates health information by using an influence of a product included in the purchase history on health of a customer. For example, the health information generating unit 130 determines an ingredient contained in a food material and/or a product included in the purchase history. This determination is performed, for example, by using the database indicating ingredients contained in each food material and each product. Further, the health information generating unit 130 determines, by using the attribute information, an ingredient that a customer should not consume. This determination of an ingredient is performed by using a database indicating an ingredient that should not be consumed for each disease name (or for each symptom, or for each physical constitution). Then, when a food material and/or a food material containing the determined ingredient is included in the purchase history, the health information generating unit 130 adds information indicating a material and/or the food material to the health information. As one example, when the attribute information indicates high blood pressure, the health information includes information indicating a food material and/or a product containing excessive amount of salt. Note that, the health information generating unit 130 may add information indicating that the ingredient that a customer should not consume is included to the health information.

Further, the health information generating unit 130 may generate the health information by using the weather information. In this case, the health information includes an estimation result of a future change in health state. As one example, the health information generating unit 130 acquires weather information for a location of the prescription information acquiring apparatus 50 (specifically, an area where a customer is likely to live). Then, when the weather information indicates, for example, that atmospheric pressure will drop (for example, low atmospheric pressure will come) a next day (or several days later), and the health information indicates that a customer is prone to headaches as a physical constitution, the health information generating unit 130 adds information indicating that a headache may occur the next day (or several days later) to the health information.

Each of pieces of the processing performed by the health information generating unit 130, for example, the generation processing of health information and the generation processing of recommendation information, may be performed by using a model generated by machine learning, or may be performed by using a program prepared in accordance with a rule base.

Then, the health information generating unit 130 transmits the generated health information to the personal terminal 20 (step S130).

Note that, the health information generating apparatus 10 may perform the processing illustrated in the present drawing at the timing desired by a customer. In this case, a customer operates the personal terminal 20, and transmits customer identification information of the customer to the health information generating apparatus 10. Then, the health information generating apparatus 10 reads, in step S100, various pieces of information associated with the customer identification information, and thereafter, performs the processing after step S110. In this processing, when the attribute information is stored in the personal information storage unit 30, the health information generating apparatus may read the attribute information from the personal information storage unit 30, instead of performing step S100 and step S110.

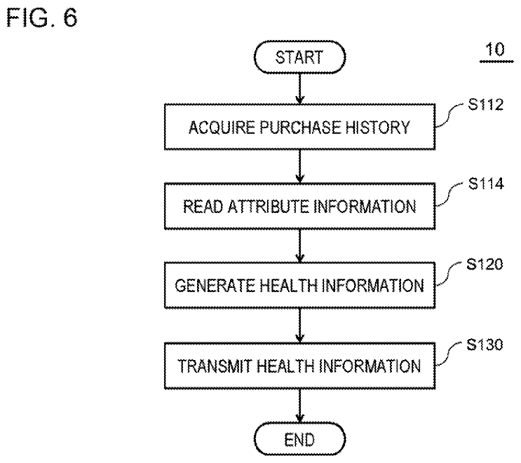

FIG. 6 is a diagram illustrating a modification example of FIG. 5. In the example illustrated in the present drawing, the health information generating apparatus 10 performs processing illustrated in the present drawing, at timing when a new purchase history is stored in the personal information storage unit 30. In this case, a most recent purchase history may not indicate an actual completed purchase. For example, the processing illustrated in the present drawing may be performed at the timing when a product is registered for payment.

First, the personal information storage unit 30 transmits a new purchase history and customer identification information associated with the new purchase history to the health information generating apparatus 10 (step S112). Next, the health information generating apparatus 10 acquires various pieces of information associated with the customer identification information acquired in step S112. The information acquired herein includes at least attribute information (step S114).

Next, the health information generating unit 130 generates health information (step S120), and transmits the generated health information to the personal terminal 20 (step S140).

Thus, according to the present example embodiment, the health information generating apparatus 10 generates attribute information of a customer by using prescription information, and generates health information by using the attribute information. The health information is information on health of the customer. Therefore, by using the health information generating apparatus 10, information beneficial to a customer can be generated from prescription information.

While the example embodiment of the present invention has been described with reference to the drawings, the example embodiment is an example of the present invention, and various configurations other than those described above maybe adopted.

Further, in the plurality of flowcharts used in the above description, a plurality of steps (pieces of processing) are described in order, but an order in which the steps are executed in each example embodiment is not limited to the order in which the steps are described. In each example embodiment, the illustrated order of the steps may be changed to an extent that contents of the processes are not interfered with. Further, each of the above-described example embodiments may be combined to an extent that contents thereof do not conflict.

A part or the entirety of the above-described example embodiment may be described as the following supplementary notes, but is not limited thereto.
1. A health information generating apparatus including:
   an acquiring unit that acquires prescription information being information described in a prescription for a customer;
   an attribute information generating unit that generates, by using the prescription information, attribute information indicating an attribute of the customer; and
   a health information generating unit that generates, by using the attribute information, health information being information on health of the customer, and outputs the health information.
2. The health information generating apparatus according to supplementary note 1, in which
   the health information relates to at least one of maintenance, improvement, and future change in health of the customer.
3. The health information generating apparatus according to supplementary note 1 or 2, in which
   the attribute information includes information indicating a health state of the customer.
4. The health information generating apparatus according to any one of supplementary notes 1 to 3, in which
   the health information generating unit generates the health information by further using a purchase history indicating an item purchased by the customer.
5. The health information generating apparatus according to supplementary note 4, in which
   the health information generating unit generates the health information by using an influence of a product included in the purchase history on health of the customer, by using the attribute information.
6. The health information generating apparatus according to any one of supplementary notes 1 to 5, in which
   the health information generating unit generates the health information by further using weather information.
7. The health information generating apparatus according to supplementary note 6, in which
   the health information includes information indicating a possible influence of weather on health of the customer.
8. The health information generating apparatus according to any one of supplementary notes 1 to 7, in which
   the health information generating unit generates the health information by further using a medical history of the customer.
9. The health information generating apparatus according to any one of supplementary notes 1 to 8, in which
   the health information generating unit generates the health information by further using a medication history of the customer.
10. The health information generating apparatus according to any one of supplementary notes 1 to 9, in which
   the health information generating unit
      estimates, by using at least one of the prescription information and the attribute information, a disease that may occur to the customer in a future, and
      generates, by using the disease, recommendation information indicating at least one of an exercise that the customer should take and a diet that the customer should consume, and outputs the generated recommendation information.
11. A health information generating method including,
   performing by a computer:
   acquiring processing of acquiring prescription information being information described in a prescription for a customer;
   attribute information generating processing of generating, by using the prescription information, attribute information indicating an attribute of the customer; and
   health information generating processing of generating, by using the attribute information, health information being information on health of the customer, and outputting the health information.
12. The health information generating method according to supplementary note 11, in which
   the health information relates to at least one of maintenance, improvement, and future change in health of the customer.
13. The health information generating method according to supplementary note 11 or 12, in which
   the attribute information includes information indicating a health state of the customer.
14. The health information generating method according to any one of supplementary notes 11 to 13, in which
   in the health information generating processing, the health information is generated by further using a purchase history indicating an item purchased by the customer.
15. The health information generating method according to supplementary note 14, in which
   in the health information generating processing, the health information is generated by using an influence of a product included in the purchase history on health of the customer, by using the attribute information.
16. The health information generating method according to any one of supplementary notes 11 to 15, in which
   in the health information generating processing, the health information is generated by further using weather information.
17. The health information generating method according to supplementary note 16, in which
   the health information includes information indicating a possible influence of weather on health of the customer.
18. The health information generating method according to any one of supplementary notes 11 to 17, in which
   in the health information generating processing, the health information is generated by further using a medical history of the customer.
19. The health information generating method according to any one of supplementary notes 11 to 18, in which
   in the health information generating processing, the health information is generated by further using a medication history of the customer.
20. The health information generating method according to any one of supplementary notes 11 to 19, in which
   in the health information generating processing:
      a disease that may occur to the customer in a future is estimated by using at least one of the prescription information and the attribute information; and
      recommendation information indicating at least one of an exercise that the customer should take and a diet that the customer should consume is generated by using the disease, and the generated recommendation information is output.
21. A program causing a computer to include:
   an acquiring function of acquiring prescription information being information described in a prescription for a customer;
   an attribute information generating function of generating, by using the prescription information, attribute information indicating an attribute of the customer; and
   a health information generating function of generating, by using the attribute information, health information being information on health of the customer, and outputting the health information.
22. The program according to supplementary note 21, in which,
   the health information relates to at least one of maintenance, improvement, and future change in health of the customer.
23. The program according to supplementary note 21 or 22, in which
   the attribute information includes information indicating a health state of the customer.
24. The program according to any one of supplementary notes 21 to 23, in which
   the health information generating function generates the health information by further using a purchase history indicating an item purchased by the customer.
25. The program according to supplementary note 24, in which
   the health information generating function generates the health information by using an influence of a product included in the purchase history on health of the customer, by using the attribute information.
26. The program according to any one of supplementary notes 21 to 25, in which
   the health information generating function generates the health information by further using weather information.
27. The program according to supplementary note 26, in which,
   the health information includes information indicating a possible influence of weather on health of the customer.
28. The program according to any one of supplementary notes 21 to 27, in which
   the health information generating function generates the health information by further using a medical history of the customer.
29. The program according to any one of supplementary notes 21 to 28, in which
   the health information generating function generates the health information by further using a medication history of the customer.
30. The program according to any one of supplementary notes 21 to 29, in which
   the health information generating function
      estimates, by using at least one of the prescription information and the attribute information, a disease that may occur to the customer in a future, and
      generates, by using the disease, recommendation information indicating at least one of an exercise that the customer should take and a diet that the customer should consume, and outputs the generated recommendation information.

### REFERENCE SIGNS LIST

10 Health information generating apparatus
20 Personal terminal
30 Personal information storage unit
40 Weather information storage unit
110 Acquiring unit
120 Attribute information generating unit
130 Health information generating unit
140 Medication information storage unit

What is claimed is:

1. A health information generating apparatus comprising:
at least one memory configured to store instructions; and
at least one processor configured to execute the instructions to perform operations, the operations comprising:
acquiring prescription information being information described in a prescription for a customer;
generating, by using the prescription information, attribute information indicating an attribute of the customer;
generating, by using the attribute information, health information being information on health of the customer; and
outputting the health information.

2. The health information generating apparatus according to claim 1, wherein
the health information relates to at least one of maintenance, improvement, and future change in health of the customer.

3. The health information generating apparatus according to claim 1, wherein
the attribute information includes information indicating a health state of the customer.

4. The health information generating apparatus according to claim 1, wherein
the operations comprise generating the health information by further using a purchase history indicating a product purchased by the customer.

5. The health information generating apparatus according to claim 4, wherein
the operations comprise generating the health information by using an influence of a product included in the purchase history on health of the customer, by using the attribute information.

6. The health information generating apparatus according to claim 1, wherein
the operations comprise generating the health information by further using weather information.

7. The health information generating apparatus according to claim 6, wherein
the health information includes information indicating a possible influence of weather on health of the customer.

8. The health information generating apparatus according to claim 1, wherein
the operations comprise generating the health information by further using a medical history of the customer.

9. The health information generating apparatus according to claim 1, wherein
the operations comprise generating the health information by further using a medication history of the customer.

10. The health information generating apparatus according to claim 1, wherein
the operations comprise
estimating, by using at least one of the prescription information and the attribute information, a disease that may occur to the customer in a future, and
generating, by using the disease, recommendation information indicating at least one of an exercise that the customer should take and a diet that the customer should consume, and outputting the generated recommendation information.

11. A health information generating method performed by a computer, the health information generating method comprising:
of acquiring prescription information being information described in a prescription for a customer;
generating, by using the prescription information, attribute information indicating an attribute of the customer;
generating, by using the attribute information, health information being information on health of the customer; and
outputting the health information.

12. A non-transitory computer-readable medium storing a program for causing a computer to perform operations, the operations comprising:
acquiring prescription information being information described in a prescription for a customer;
generating, by using the prescription information, attribute information indicating an attribute of the customer;
generating, by using the attribute information, health information being information on health of the customer; and
outputting the health information.
